(19) United States
(12) Patent Application Publication
(10) Pub. No.: US 2011/0130573 A1
(43) Pub. Date: Jun. 2, 2011

(54) METHOD FOR PRODUCING PHTHALIC ACID ANHYDRIDE

(75) Inventors: Evin Hizaler Hoffmann, Koln (DE); Leslaw Mleczko, Dormagen (DE); Ralph Schellen, Dormagen (DE); Stephan Schubert, Leverkusen (DE); André Hoffmann, Munchen (DE)

(73) Assignee: Bayer Technology Services GMBH, Leverkusen (DE)

(21) Appl. No.: 12/991,290

(22) PCT Filed: May 26, 2009
(86) PCT No.: PCT/EP09/03717

§ 371 Date: Feb. 16, 2011

(30) Foreign Application Priority Data
May 29, 2008 (DE) 10 2008 025 843.1

(51) Int. Cl. C07D 307/89; B01J 8/04

(52) U.S. Cl. 549/248; 422/636

(57) ABSTRACT

Method for the production of phthalic acid anhydride by catalytic gas phase oxidation of o-xylol with oxygen, in which the reaction is carried out in 5 to 60 serially arranged reaction zones under adiabatic conditions, and reactor system for carrying out the method.

The present invention relates to a process for preparing phthalic anhydride by catalytic gas-phase oxidation of o-xylene by means of oxygen, wherein the reaction is carried out in from 5 to 60 reaction zones connected in series under adiabatic conditions, and also a reactor system for carrying out the process.

Phthalic anhydride is generally prepared from gaseous o-xylene and oxygen in the presence of metal oxide catalysts, e.g. vanadium pentoxide, in an exothermic, catalytic reaction according to formula (I):

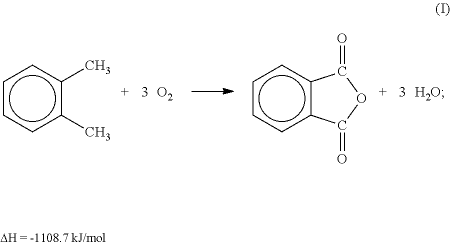

The phthalic anhydride prepared by means of the reaction according to formula (I) is frequently used as starting material for the preparation of plasticizers (usually phthalic esters) or as raw material for producing synthetic resins for surface coatings on wood. In addition, it is a raw material in the preparation of dyes or pigments based on phthalocyanines. A further industrially important reaction of phthalic anhydride is that to form anthraquinone.

The removal and use of the heat of reaction is important in carrying out the phthalic anhydride synthesis. An uncontrolled temperature rise can lead to permanent damage to the catalyst. In addition, the possibility of secondary reactions to form more or less large amounts of maleic anhydride and also carbon monoxide and carbon dioxide exists at high temperatures. It is therefore advantageous to control the temperature of the reaction zones during the process so as to keep them at a level which allows a rapid reaction with minimization of the secondary reactions and/or catalyst deactivation.

U.S. Pat. No. 6,380,399 B1 discloses a process for preparing phthalic anhydride from o-xylene or naphtha, in which the reaction is carried out at temperatures in the range from 300° C. to 400° C. in at least three catalyst stages, which can be combined in one reaction zone, in a fixed-bed reactor. The proportion of oxygen in the process gases entering the process is in the range from 10 to 21% by volume. Based on this, the concentration of the o-xylene or naphtha is at least 70 g/standard m³. The catalyst in whose presence the reaction is carried out preferably comprises vanadium oxide and titanium oxide. It is further disclosed that the reaction zones are cooled. No separate heat exchanger zones are disclosed.

The process disclosed in U.S. Pat. No. 6,380,399 B1 is disadvantageous because the reaction zones are actively cooled and an attempt is made to prevent overheating of the reaction zones by adjusting the catalyst activity by dilution with material which is not catalytically active. The fact that the reaction zones are cooled directly leads to only a maximum heat exchange rate prescribed by the geometry of the reaction apparatus and the heat transfer medium used being able to be achieved and not being able to be changed in a simple manner during operation of the process. This also results in operating states in which superheating of the process in the “hot spots” can no longer be prevented from being able to occur, e.g. as a result of too much starting material being fed in because of incorrect setting of a valve. This leads at least to a reduced yield of the target product but can also lead to a decrease in catalyst activity and thus to the necessity of replacing the catalyst, or to destruction of the apparatus in which the reaction zone is located.

U.S. Pat. No. 6,774,246 B2 discloses a process similar to the disclosure of U.S. Pat. No. 6,380,399 B1, in which phthalic anhydride is prepared from o-xylene or naphtha in two reaction zones, where the reaction zones comprise fixed beds which are cooled. The total pressure in the process can be in the range from 0.1 to 2.5 bar, while the temperatures at which the process gases enter the process can be in the range from 300° C. to 450° C. The process gas can comprise from 1 to 100 mol % of oxygen and have a concentration of o-xylene or naphtha in the range from 60 to 120 g/standard m³. As in U.S. Pat. No. 6,380,399 B1 too, no heat exchange zones separate from the reaction zones are disclosed.

Accordingly, the process disclosed in U.S. Pat. No. 6,774,246 B2 is disadvantageous for the same reasons as the process disclosed in U.S. Pat. No. 6,380,399 B1. In addition, the use of not more than two reactions zones results in still poorer temperature control.

EP 1 251 951 (B1) discloses an apparatus and the opportunity of carrying out chemical reactions in the apparatus, where the apparatus is characterized by a cascade of reaction zones and heat exchange apparatuses which are in contact with one another and are integrated with one another in terms of material. The process to be carried out therein is thus characterized by contact of the various reaction zones with a respective heat exchange apparatus in the form of a cascade. A disclosure in respect of the usability of the apparatus and of the process for the synthesis of phthalic anhydride from gaseous oxygen and o-xylene is not to be found. It therefore remains unclear how, proceeding from the disclosure of EP 1 251 951 (B1), such a reaction can be carried out by means of the apparatus and the process carried out therein. Furthermore, for reasons of unity, it has to be assumed that the process disclosed in EP 1 251 951 (B1) is carried out in an apparatus identical or similar to the disclosure in respect of the apparatus. As a result, due to the large-area contact of the heat exchange zones with the reaction zones as per the disclosure, a significant amount of heat is transferred by thermal conduction between the reaction zones and the adjacent heat exchange zones. The disclosure in respect of the oscillating temperature profile can thus only be interpreted as meaning that the temperature peaks found here would be larger if this contact did not exist. A further indication of this is the exponential rise in the disclosed temperature profiles between the individual temperature peaks. These indicate that some heat sink which has an appreciable but limited capacity and can reduce the temperature rise is present in each reaction zone. It can never be ruled out that some removal of heat (e.g. by radiation) takes place, but a reduction in the possible heat removal from the reaction zone would be indicated by a linear temperature profile or a temperature profile having a degressive gradient, since no further introduction of starting materials is provided and after an exothermic reaction, the reaction would proceed ever more slowly and thus with a reduced evolution of heat. Thus, EP 1 251 951 (B1) discloses multistage processes in cascades of reaction zones from which heat is removed in an undefined amount by thermal conduction. Accordingly, the process disclosed has the disadvantage that precise temperature control of the process gases of the reaction is not possible.

Proceeding from the prior art, it would therefore be advantageous to provide a process which can be carried out in simple reaction apparatuses and allows precise, simple temperature control so that it allows high conversions at very high product purities. Such simple reaction apparatuses would be simple to scale up to an industrial scale and are inexpensive and robust in all sizes.

As just indicated, neither suitable reactors nor suitable processes which allow these objectives to be achieved have hitherto been described for the catalytic gas-phase oxidation of o-xylene by means of oxygen to form phthalic anhydride.

It is therefore an object of the invention to provide a process for the catalytic gas-phase oxidation of o-xylene by means of oxygen to form phthalic anhydride, which process can be carried out with precise temperature control in simple reaction apparatuses and thus allows high conversions at high product purities, with the heat of reaction being able to be utilized to the benefit of the reaction or in another way.

It has surprisingly been found that a process for preparing phthalic anhydride from o-xylene and oxygen in the presence of heterogeneous catalysts, characterized in that it comprises from 5 to 60 reaction zones which are connected in series and have adiabatic conditions, is able to achieve this object.

The term o-xylene refers, in the context of the present invention, to a process gas which is introduced into the process of the invention and comprises o-xylene. The proportion of o-xylene in the process gases fed to the process is usually in the range from 0.8 to 10 mol %, preferably from 1 to 7 mol %.

The term oxygen refers, in the context of the present invention, to a process gas which is introduced into the process of the invention and comprises essentially oxygen. Oxygen is preferably ambient air and therefore has a proportion of about 20% by volume of oxygen.

Apart from the essential components of the process gases o-xylene and oxygen, these gases can also comprise secondary components. Nonexhaustive examples of secondary components which can be present in the process gases are, for instance, argon, nitrogen and/or carbon dioxide.

In general, process gases are, in the context of the present invention, gas mixtures which comprise oxygen and/or o-xylene and/or phthalic anhydride and/or secondary components.

For the purposes of the invention, carrying out the process under adiabatic conditions means that essentially no heat is either actively introduced or actively removed from the reaction zone from or to the outside. It is generally known that complete insulation against introduction or removal of heat can be achieved only by complete evacuation and ruling out heat transfer by radiation. Therefore, in the context of the present invention, adiabatic means that no measures for introducing or removing heat are taken.

In an alternative embodiment of the process of the invention, heat transfer can be reduced by, for example, insulation by means of generally known insulation materials, e.g. polystyrene insulation materials, or by means of sufficiently large distances to heat sinks or heat sources, with the insulation material being air.

An advantage of the adiabatic mode of operation according to the invention of the 5 to 60 reaction zones connected in series over a nonadiabatic mode of operation is that no means of removing heat have to be provided in the reaction zones, which results in a considerable simplification of the construction. Simplifications in the manufacture of the reactor and also in the scalability of the process and an increase in the reaction conversions are, in particular, obtained in this way. In addition, the heat generated during the course of the exothermic reaction is utilized in a controlled manner in the individual reaction zone to increase the conversion.

A further advantage of the process of the invention is the possibility of very precise temperature control by means of the close spacing of adiabatic reaction zones. It is thus possible for a temperature advantageous to the progress of the reaction to be set and controlled in each reaction zone.

The catalysts used in the process of the invention are usually catalysts comprising a material which not only have catalytic activity for the reaction according to formula (I) but are also characterized by sufficient chemical resistance under the conditions of the process and also by a high specific surface area. Catalyst materials which are characterized by such a chemical resistance under the conditions of the process are, for example, catalysts comprising mixed oxides of vanadium and also oxides and/or salts of elements selected from the list of elements consisting of Nb, Sb, P, K, Na, Cs, Rb and Mo. These are preferably catalysts comprising mixed oxides of vanadium with P and Rb. These catalysts can be applied to support materials. Such support materials usually comprise aluminum oxide, silicon dioxide and/or titanium dioxide. Preference is given to support materials composed of titanium dioxide.

The term specific surface area refers, in the context of the present invention, to the area of the catalyst material which can be reached by the process gas, based on the mass of catalyst material used.

A high specific surface area is a specific surface area of at least 10 m²/g, preferably at least 20 m²/g.

The catalysts used according to the invention are in each case located in the reaction zones and can be present in all forms known per se, e.g. fixed bed, moving bed, fluidized bed.

Preference is given to fixed beds and moving beds.

The fixed-bed arrangement comprises a catalyst bed in the actual sense, i.e. loose, supported or unsupported catalyst of any shape, and also in the form of suitable packings. The term catalyst bed as used here also encompasses contiguous regions of suitable packings on a support material or structured catalyst support. These would be, for example, ceramic honeycomb bodies having comparatively high geometric surface areas to be coated or corrugated layers of woven metal wire mesh on which, for example, catalyst granules are immobilized. In the context of the present invention, a special form of packing is the presence of the catalyst in monolithic form.

If a fixed-bed arrangement of the catalyst is used, the catalyst is preferably present in beds of particles having average particle sizes of from 1 to 10 mm, preferably from 2 to 8 mm, particularly preferably from 4 to 7 mm.

Preference is likewise given to the catalyst in a fixed-bed arrangement being present in monolithic form. A particularly preferred embodiment of a fixed-bed arrangement is a monolithic catalyst comprising mixed oxides of vanadium with phosphorus supported on titanium dioxide.

If a catalyst in monolithic form is used in the reaction zones, the catalyst present in monolithic form is, in a preferred embodiment of the invention, provided with channels through which the process gases flow. The channels usually have a diameter of from 0.1 to 3 mm, preferably a diameter of from 0.2 to 2 mm, particularly preferably from 0.5 to 1.5 mm.

A monolithic catalyst having channels of the diameter indicated is particularly advantageous since protection against explosion can be ensured thereby. This is achieved by uptake of the enthalpy by the wall of the monolith, as a result of which the spread of flames is suppressed.

If a moving-bed arrangement of the catalyst is used, the catalyst is preferably present in loose beds of particles as have been described above in connection with the fixed-bed arrangement.

Beds of such particles are advantageous because the particles of such a size have a high specific surface area of the catalyst material toward the process gases oxygen and o-xylene and a high reaction rate can therefore be achieved. The mass transfer limitation of the reaction by diffusion can thus be kept low. At the same time, the particles are not yet so small that disproportionally increased pressure drops occur on flow through the fixed bed. The ranges of the particle sizes indicated in the preferred embodiment of the process comprising a reaction in a fixed bed are thus an optimum between the achievable conversion in the reaction according to formula (I) and the pressure drop produced when carrying out the process. The pressure drop is coupled directly to the energy required in the form of compressor power, so that a disproportionate increase in the latter would result in uneconomical operation of the process.

In a preferred embodiment of the process of the invention, the reaction is carried out in from 6 to 30, particularly preferably from 7 to 20, reaction zones connected in series.

A preferred further embodiment of the process is characterized in that the process gas leaving at least one reaction zone is subsequently passed through at least one heat exchange zone located downstream of this reaction zone.

In a particularly preferred further embodiment of the process, each reaction zone is followed by at least one, preferably precisely one, heat exchange zone through which the process gas leaving the reaction zone is passed.

The reaction zones can either be arranged in one reactor or be divided between a plurality of reactors. The arrangement of the reaction zones in one reactor leads to a reduction in the number of apparatuses used.

The individual reaction zones and heat exchange zones can also be arranged together in one reactor or in any combinations of reaction zones with heat exchange zones in a plurality of reactors.

If reaction zones and heat exchange zones are present in one reactor, a thermal insulation zone is, in an alternative embodiment of the invention, present between these in order to be able to maintain adiabatic operation of the reaction zone.

In addition, individual reaction zones among the reaction zones connected in series can also, independently of one another, be replaced or supplemented by one or more reaction zones connected in parallel. The use of reaction zones connected in parallel allows, in particular, replacement or supplementation of these during ongoing continuous overall operation of the process.

Parallel reaction zones and reaction zones connected in series can, in particular, also be combined with one another. However, the process of the invention particularly preferably has exclusively reaction zones connected in series.

The reactors which are preferably used in the process of the invention can comprise simple vessels having one or more reaction zones, as are described, for example, in Ullmanns Encyclopedia of Industrial Chemistry (Fifth, Completely Revised Edition, Vol B4, pages 95-104, pages 210-216), with thermal insulation zones being able to be additionally provided in each case between the individual reaction zones and/or heat exchange zones.

In an alternative embodiment of the process, at least one thermal insulation zone is thus located between a reaction zone and a heat exchange zone. Preference is given to a thermal insulation zone being present around each reaction zone.

The catalysts or the fixed beds of catalysts are applied in a manner known per se to or between gas-permeable walls comprising the reaction zone of the reactor. Particularly in the case of thin fixed beds, technical devices for obtaining uniform distribution of gas can be installed upstream of the catalyst beds. These can be perforated plates, bubble cap trays, valve trays or other internals which, by producing a small but uniform pressure drop, bring about uniform entry of the process gas into the fixed bed.

In a preferred embodiment of the process, the entry temperature of the process gas entering the first reaction zone is from 10 to 490° C., preferably from 150 to 480° C., particularly preferably from 300 to 470° C.

In a further preferred embodiment of the process, the absolute pressure at the entrance into the first reaction zone is in the range from 1 to 10 bar, preferably from 1.1 to 3 bar, particularly preferably from 1.2 to 1.5 bar.

In another preferred embodiment of the process, the residence time of the process gas in all reaction zones together is in the range from 0.05 to 25 s, preferably from 0.1 to 10 s, particularly preferably from 0.15 to 3 s.

The o-xylene and the oxygen are preferably fed in only upstream of the first reaction zone. This has the advantage that the entire process gas can be utilized for taking up and removing the heat of reaction in all reaction zones. In addition, such a mode of operation enables the space-time yield to be increased or the mass of catalyst necessary to be reduced. However, it is also possible to introduce o-xylene and/or oxygen into the process gas as required before one or more of the reaction zones following the first reaction zone. The introduction of gas between the reaction zones additionally allows the temperature of the reaction to be controlled.

In a preferred embodiment of the process of the invention, the process gas is cooled after at least one of the reaction zones used, particularly preferably after each reaction zone. For this purpose, the process gas leaving a reaction zone is passed through one or more of the abovementioned heat exchange zones which are located downstream of the respective reaction zones. These can be configured as heat exchange zones in the form of the heat exchangers known to those skilled in the art, e.g. shell-and-tube, plate, annular groove, spiral, finned tube, micro heat exchangers. The heat exchangers are preferably microstructured heat exchangers.

The term microstructured means, in the context of the present invention, that the heat exchanger has, for the purposes of heat transfer, fluid-conducting channels which are characterized in that they have a hydraulic diameter in the range from 50 μm to 5 mm. The hydraulic diameter is given by four times the cross-sectional area of the fluid-conducting channel through which flow occurs divided by the circumference of the channel.

In a particular embodiment of the process, steam is generated by the heat exchanger during cooling of the process gas in the heat exchange zones.

Within this particular embodiment, preference is given to carrying out a vaporization, preferably partial vaporization, on the side of the cooling medium in the heat exchangers comprising the heat exchange zones.

In the context of the present invention, partial vaporization is vaporization in which a gas/liquid mixture of a substance is used as cooling medium and in which a gas/liquid mixture of a substance is still present after heat transfer in the heat exchanger.

Carrying out a vaporization is particularly advantageous because the achievable heat transfer coefficient from/to process gases to/from cooling/heating medium becomes particularly high as a result and efficient cooling can therefore be achieved.

The carrying out of a partial vaporization is particularly advantageous because the uptake/release of heat by the cooling medium then no longer results in a temperature change in the cooling medium but only produces a shift in the gas/liquid equilibrium. As a result, the process gas is cooled against a constant temperature over the entire heat exchange zone. This in turn reliably prevents occurrence of temperature profiles in the flow of the process gases, as a result of which control over the reaction temperatures in the reaction zones is improved and, in particular, the formation of local hot spots due to temperature profiles is prevented.

In an alternative embodiment, a mixing zone can be provided instead of a vaporization/partial vaporization before the entrance to a reaction zone in order to even out any temperature profiles in the flow of the process gases arising during cooling by mixing transverse to the main flow direction.

In a preferred embodiment of the process, the reaction zones connected in series are operated at an average temperature which increases or decreases from reaction zone to reaction zone. This means that, within a sequence of reaction zones, the temperature can both increase and decrease from reaction zone to reaction zone. This can be achieved, for example, by control of the heat exchange zones located between the reaction zones. Further possibilities for setting the average temperature are described below.

The thickness of the reaction zones through which flow occurs can be made identical or different and is derived according to laws generally known to those skilled in the art from the above-described residence time and the amounts of process gas put through the process in each case. The mass flows of product gas (phthalic anhydride) which can be put through the process according to the invention, from which the amounts of process gas to be used are also derived, are usually in the range from 0.01 to 35 t/h, preferably from 0.1 to 20 t/h, particularly preferably from 1 to 15 t/h.

The maximum exit temperature of the process gas from the reaction zones is usually in the range from 400° C. to 520° C., preferably from 420° C. to 510° C., particularly preferably from 430° C. to 500° C. The control of the temperature in the reaction zones is preferably effected by means of at least one of the following measures: dimensioning of the adiabatic reaction zone, control of the heat removal between the reaction zones, addition of gas between the reaction zones, molar ratio of the starting material/excess of oxygen used, addition of inert gases, in particular nitrogen, carbon dioxide, before and/or between the reaction zones.

The composition of the catalysts in the reaction zones according to the invention can be identical or different. In a preferred embodiment, the same catalysts are used in each reaction zone. However, different catalysts can also advantageously be used in the individual reaction zones. Thus, it is possible, in particular, to use a less active catalyst in the first reaction zone where the concentration of the reactants is still high and to increase the activity of the catalyst from reaction zone to reaction zone in the further reaction zones. The catalyst activity can also be controlled by dilution with inert materials or support material. The use of a catalyst which is particularly stable toward deactivation at the temperatures of the process in the first and/or second reaction zones in these reactions zones is likewise advantageous.

The process of the invention makes it possible to produce, per 1 kg of catalyst, from 0.01 kg/h to 1 kg/h, preferably from 0.02 kg/h to 0.75 kg/h, particularly preferably from 0.03 kg/h to 0.4 kg/h, of phthalic anhydride.

The process of the invention is thus characterized by high space-time yields, combined with a reduction in the sizes of the apparatuses and a simplification of the apparatuses or reactors. This surprisingly high space-time yield is made possible by interaction of the inventive and preferred embodiments of the novel process. In particular, the interaction of gradated, adiabatic reaction zones with heat exchange zones located between them and the defined residence times makes possible precise control of the process and the resulting high space-time yields and also a reduction in the by-products formed, e.g. maleic anhydride and CO₂.

The invention further provides a reactor system for reacting o-xylene and oxygen to form phthalic anhydride, characterized in that it comprises feed lines (Z) for a process gas comprising o-xylene and oxygen or for at least two process gases of which at least one comprises o-xylene and at least one comprises oxygen and comprises from 5 to 60 reaction zones (R) which are connected in series and are in the form of fixed beds of a heterogeneous catalyst, where thermal insulation zones (I) in the form of insulation material are located between the reaction zones and heat exchange zones (W) in the form of plate heat exchangers which are connected to the reaction zones via feed lines and discharge lines for the process gases and comprise feed lines and discharge lines for a cooling medium are located between these thermal insulation zones.

The reactor system can also comprise from 6 to 30, preferably from 7 to 20, reaction zones in the form of fixed beds.

The insulation material of the thermal insulation zones is preferably a material having a coefficient of thermal conductivity λ less than or equal to 0.08

$$[ \frac{W}{m \cdot K} ] .$$

Particular preference is given to, for instance, polystyrene, polyurethanes, glass wool or air.

The present invention will be illustrated with the aid of the drawings, but is not restricted thereto.

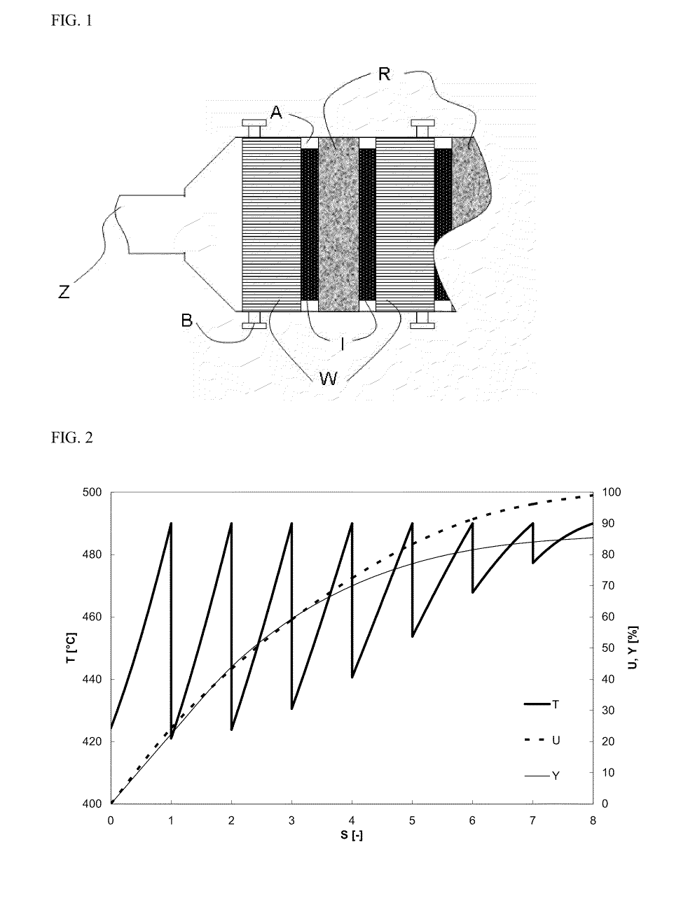

FIG. 1 schematically shows an embodiment of the reactor system of the invention, where the following reference numerals are used in the drawing:
Z: feed line(s)
R: reaction zone(s)
I: thermal insulation zone(s)
W: heat exchange zone(s)

FIG. 2 shows reactor temperature (T), o-xylene conversion (U) and phthalic anhydride selectivity (Y) over a number of 8 reaction zones (S) followed by heat exchange zones (as per example 1).

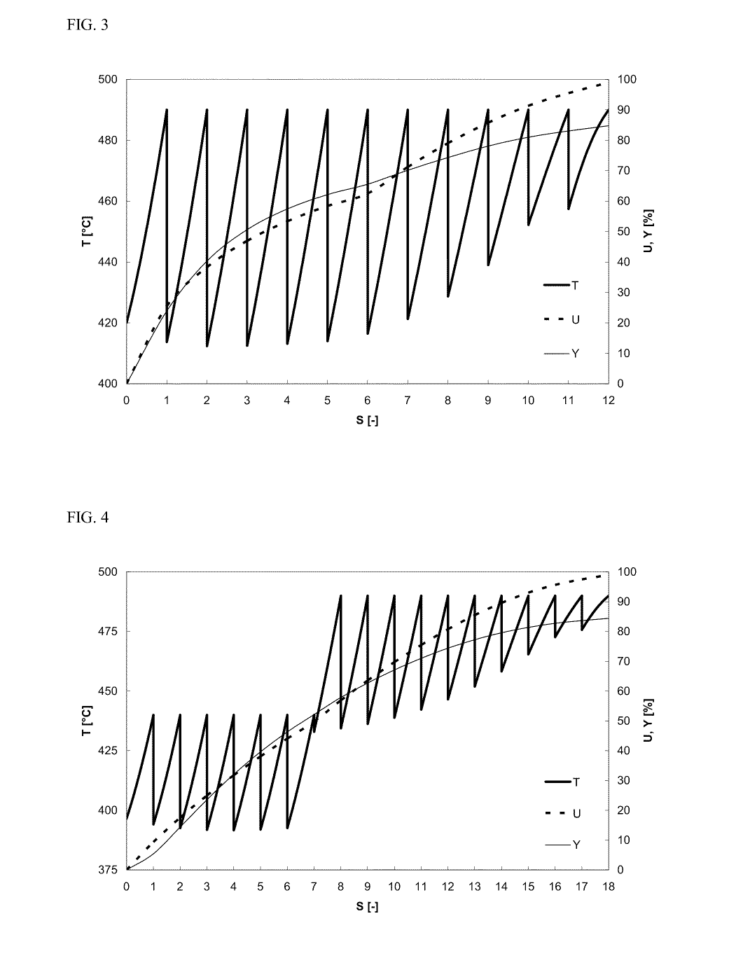

FIG. 3 shows reactor temperature (T), o-xylene conversion (U) and phthalic anhydride selectivity (Y) over a number of 12 reaction zones (S) followed by heat exchange zones (as per example 2).

FIG. 4 shows reactor temperature (T), o-xylene conversion (U) and phthalic anhydride selectivity (Y) over a number of 18 reaction zones (S) followed by heat exchange zones (as per example 3).

The present invention will also be illustrated by examples 1 to 3 below, without being restricted thereto.

### EXAMPLES

### Example 1

In this example, the process gas flows through a total of 8 fixed catalyst beds composed of titanium dioxide coated with vanadium pentoxide, i.e. through 8 reaction zones. After each reaction zone, there is a heat exchange zone in which the process gas was cooled before entering the next reaction zone. The process gas used at the entry to the first reaction zone contains 0.94 mol % of o-xylene, 20.79 mol % of oxygen and 78.27 mol % of inert gases (nitrogen, CO₂, argon). With a proportion of 0.94 mol % of o-xylene, the proportion is below the limit for obtaining a potentially ignitable mixture (1 mol % with air), so that it is not necessary to be concerned about exceeding a potential ignition temperature. The absolute entry pressure of the process gas directly before the first reaction zone is 1.5 bar. The length of the fixed catalyst beds, i.e. the reaction zones, is in each case 0.1 m except for the last reaction zone whose length is 0.14 m. The activity of the catalyst used cannot be changed over the reaction zones. No gas is introduced before the individual reaction zones. The total residence time in the plant is 0.2 seconds.

The results are shown in FIG. 2. Here, the individual reaction zones are shown on the x axis, so that a spatial course of the developments in the process can be seen. The temperature of the process gas is indicated on the left-hand y axis. The course of the temperature over the individual reaction zones is shown as a bold, solid line. The total conversion of o-xylene and the selectivity to phthalic anhydride is indicated on the right-hand y axis. The course of the conversion over the individual reaction zones is shown as a bold broken line. The course of the selectivity is shown as a thin solid line.

It can be seen that the entry temperature of the process gas before the first reaction zone is about 420° C. As a result of the exothermic reaction to form phthalic anhydride under adiabatic conditions, the temperature rises to about 490° C. in the first reaction zone before the process gas is cooled again in the following heat exchange zone. The entry temperature before the next reaction zone is again about 420° C. As a result of the exothermic adiabatic reaction, it increases again to about 490° C. The sequence of heating and cooling continues. The entry temperatures of the process gas before the individual reaction zones changes to about 480° C. over the course of the process.

A conversion of o-xylene of 99 mol % is obtained. The selectivity obtained is 85.4 mol %. The space-time yield achieved, based on the mass of catalyst used, is 0.19 kg_{phthalic anhydride}/kg_{cat}h.

### Example 2

In this example, the process gas flows through a total of 12 reaction zones, i.e. through 12 fixed catalyst beds composed of titanium dioxide coated with vanadium pentoxide. After each reaction zone, there is a heat exchange zone in which the process gas is cooled and in which a further addition of o-xylene before reaction zones is carried out as per table 1. This addition is regulated so that the ignition limit of 1 mol % of o-xylene is not reached and it is not necessary to be concerned about exceeding a potential ignition temperature. The precise proportions of o-xylene based on the total amount introduced into the process are shown in table 1.

TABLE 1
Division of the introduction of o-xylene as per example 2

| | | Proportion of total amount |
|---|---|---|
| Designation | Addition before reaction zone | of o-xylene [%] |
| Feed | 1 | 46.6 |
| Addition 1 | 2 | 12.3 |
| Addition 2 | 3 | 10.5 |
| Addition 3 | 4 | 10.3 |
| Addition 4 | 5 | 10.3 |
| Addition 5 | 6 | 10 |

The process gas used at the beginning and also the entry pressure before the first reaction zone are identical to those in example 1. In the case of the additions, streams of pure, gaseous o-xylene are used in order to at least partly replace the amount consumed. The volumes and streams of the additions can thus be derived from the proportion shown in table 1 and the volume flow the feed and concentration of o-xylene in the feed. The length of the fixed catalyst beds, i.e. the reaction zones, is in each case 0.1 m except for the last reaction zone whose length is 0.18 m. The activity of the catalysts cannot be changed over the reaction zones. Thus, oscillation in a temperature window from 415° C. to 490° C. is achieved after the first 6 reaction zones. The total residence time in the plant is 0.3 seconds.

The results are shown in FIG. 3. Here, the individual reaction zones are shown on the x axis, so that a spatial course of the developments in the process can be seen. The temperature of the process gas is indicated on the left-hand y axis. The course of the temperature over the individual reaction zones is shown as a bold, solid line. The total conversion of o-xylene and the selectivity to phthalic anhydride is indicated on the right-hand y axis. The course of the conversion over the individual reaction zones is shown as a bold broken line. The course of the selectivity is shown as a thin solid line.

It can be seen that the entry temperature of the process gas before the first reaction zone is about 420° C. As a result of the exothermic reaction to form phthalic anhydride under adiabatic conditions, the temperature rises to about 490° C. in the first reaction zone before the process gas is cooled again in the following heat exchange zone. Cooling here is also achieved by means of the o-xylene introduced at a lower temperature. The entry temperature before the next reaction zone is again about 415° C. As a result of the exothermic adiabatic reaction, it increases again to about 490° C. The sequence of heating and cooling continues. The entry temperatures of the process gas before the individual reaction zones changes significantly from the 7th reaction zone onward. Here, further heating to about 460° C. before the last reaction zone is permitted.

A conversion of 99 mol % of the o-xylene used, calculated from the remaining mass at the exit from the last reaction zone, is obtained. The selectivity to phthalic anhydride is about 84.7 mol %. The space-time yield achieved, based on the mass of catalyst used, is 0.25 kg_{phthalic anhydride}/kg_{cat}h.

### Example 3

In this example, the process gas flows through a total of 18 fixed catalyst beds in the form of monoliths which have channel diameters of the monoliths of 1 mm and are coated with a catalyst comprising vanadium pentoxide on a titanium dioxide support, i.e. through 18 reaction zones. After each reaction zone, there is a heat exchange zone in which the process gas is cooled before it enters the next reaction zone. The process gas used at the entry into the first reaction zone contains 2 mol % of o-xylene, 20.56 mol % of oxygen and 77.44 mol % of inert gases (nitrogen, CO₂, argon). With a proportion of 2 mol % of o-xylene, the proportion is above the limit for obtaining a potentially ignitable mixture (1 mol % with air), so that care has to be taken not to exceed a potential ignition temperature (450° C.). After the seventh reaction zone, enough o-xylene has been reacted so that higher temperatures are also permitted in the further reaction zones. The absolute entry pressure of the process gas directly before the first reaction zone is 1.5 bar. The length of the fixed catalyst beds, i.e. the reaction zones, is in each case 0.5 m except for the last reaction zone which has a length of 0.74 m. The activity of the catalyst used cannot be changed over the reaction zones. No gas is introduced before the individual reaction zones. The total residence time in the plant is 1.6 seconds.

The results are shown in FIG. 4. Here, the individual reaction zones are shown on the x axis, so that a spatial course of the developments in the process can be seen. The temperature of the process gas is indicated on the left-hand y axis. The course of the temperature over the individual reaction zones is shown as a bold, solid line. The total conversion of o-xylene and the selectivity to phthalic anhydride is indicated on the right-hand y axis. The course of the conversion over the individual reaction zones is shown as a bold broken line. The course of the selectivity is shown as a thin solid line.

It can be seen that the entry temperature of the process gas before the first reaction zone is about 400° C. As a result of the exothermic reaction to form phthalic anhydride under adiabatic conditions, the temperature rises to about 440° C. in the first reaction zone before the process gas is cooled again in the following heat exchange zone. The entry temperature before the next reaction zone is about 394° C. As a result of the exothermic adiabatic reaction, it increases again to about 440° C. The sequence of heating and cooling continues to the exit of the sixth reaction zone. In the following heat exchange zone, less cooling is employed so that the entry temperature into the seventh reaction zone is about 435° C. This increases to about 490° C. as a result of the exothermic adiabatic reaction. The sequence of heating and cooling continues, with a slow rise in the entry temperatures up to 475° C. at the entry into the last reaction zone being tolerated.

A conversion of o-xylene of 99 mol % is obtained. The selectivity obtained is 84.4 mol %. The space-time yield achieved, based on the mass of catalyst used, is 0.034 kg_{phthalic anhydride}/kg_{cat}h.

1. A process for preparing phthalic anhydride by the reaction of o-xylene with oxygen in the presence of heterogeneous catalysts, wherein said process is carried out in from 5 to 60 reaction zones having adiabatic conditions, connected in series.

2. The process as claimed in claim 1, wherein the reaction is carried out in from 6 to 30 reaction zones connected in series.

3. The process as claimed in claim 1 wherein the entry temperature of the process gas entering the first reaction zone is from 10 to 490° C.

4. The process as claimed in claim 1, wherein the absolute pressure at the entry into the first reaction zone is in the range from 1 to 10 bar.

5. The process as claimed in claim 1, wherein the residence time of the process gas in all reaction zones is in the range from 0.05 to 25 seconds.

6. The process as claimed in claim 1, wherein the catalysts comprise mixed oxides of vanadium and also oxides and/or salts of elements selected from the group consisting of Nb, Sb, P, K, Na, Cs, Rb and Mo.

7. The process as claimed in claim 1, wherein the catalysts are present in a fixed bed arrangement.

8. The process as claimed in claim 7, wherein the catalysts are present as monoliths.

9. The process as claimed in claim 8, the monoliths have channels having a diameter of from 0.1 to 3 mm.

10. The process as claimed in claim 1, wherein the catalysts are present in a moving bed arrangement.

11. The process as claimed in claim 1, wherein the catalysts are present in beds of particles having average particle sizes of from 1 to 10 mm.

12. The process as claimed in claim 1, wherein at least one heat exchange zone through which the process gas is passed is present after at least one reaction zone.

13. The process as claimed in claim 12, wherein at least one heat exchange zone through which the process gas is passed is present after each reaction zone.

14. The process as claimed in claim 1, wherein at least one thermal insulation zone is present between a reaction zone and a heat exchange zone.

15. The process as claimed in claim 14, wherein a thermal insulation zone is present around each reaction zone.

16. A reactor system for carrying out the process of claim 1, comprising feed lines for a process gas comprising o-xylene and oxygen or for at least two process gases of which at least one comprises o-xylene and at least one comprises oxygen, and comprises from 5 to 60 reaction zones which are connected in series and are in the form of fixed beds of a heterogeneous catalyst, where thermal insulation zones in the form of insulation material are located between the reaction zones, and heat exchange zones in the form of plate heat exchangers which are connected to the reaction zones via feed lines and discharge lines for the process gases and comprise feed lines and discharge lines for a cooling medium are located between these thermal insulation zones.
